(19) United States
(12) Patent Application Publication
(10) Pub. No.: US 2009/0203920 A1
(43) Pub. Date: Aug. 13, 2009

(54) METHOD FOR ISOLATING 5-SUBSTITUTED TETRAZOLES

(75) Inventors: Stefan Welzig, Wien (AT); Anton Gerdenitsch, Rohrbach (AT); Wolfgang Oberleitner, Jennersdorf (AT)

Correspondence Address: POPOVICH, WILES & O'CONNELL, PA;650 THIRD AVENUE SOUTH, SUITE 600, MINNEAPOLIS, MN, 55402, US

(73) Assignee: SANOCHEMIA PHARMAZEUTIKA AG, Wien (AT)

(21) Appl. No.: 11/997,372

(22) PCT Filed: Aug. 3, 2006
(86) PCT No.: PCT/AT06/00328

§ 371 Date: Jul. 2, 2008

(30) Foreign Application Priority Data
Aug. 4, 2005 (AT) A 1317/2005

(51) Int. Cl. C07D 257/04

(52) U.S. Cl. 548/253

(57) ABSTRACT

The invention relates to a method for isolating 5-substituted tetrazoles of general formula (I)

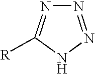

in which R represents a substituted biphenyl radical during which the ring closure, starting from a corresponding nitrile, is carried out in organic solvents while using alkali, alkaline-earth or organotin azides. The organic phases containing the nitrile and the tetrazol are firstly mixed with water while firstly forming three liquid phases, after which the aqueous phase containing the azide and the phase containing the nitrile are separated out, and the middle organic phase containing the tetrazol is subsequently processed. In the case of ester groups to be saponified, this phase is mixed with alkali lye, after which the organic phase is separated out and the aqueous phase is acidified or otherwise, this phase is immediately acidified and purified.

The invention refers to a method for isolating 5-substituted tetrazoles of general formula I:

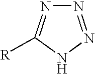

in which R represents a substituted biphenyl radical, during which ring closure, starting from a corresponding nitrile, is carried out while using in organic solvents alkali, alkaline-earth metal, or organotin azides.

5-substituted tetrazoles can be produced by the reaction of cyano compounds or nitrites with azides and in turn, in addition to HN₃, with alkali or alkaline-earth metal or organotin azides, such as trialkyl or triaryltin azides. Here, in connection with the production of sartans, EP 443983 A1 shows that the reaction with sodium or potassium azide and triethyl, tributyltin or triphenyltin azides is preferred. In particular, 5-substituted tetrazoles whose substituents represent a substituted biphenyl radical have gained attention as pharmaceuticals, wherein, above all, the group of sartans are noteworthy, such as valsartan, losartan, irbesartan, olmesartan, and candesartan. These 5-substituted tetrazoles are characterized in that in the course of the reaction starting from nitrites or cyanides to tetrazole rings, different hydrophilically or lipophilically acting substituents are present, wherein, in the case of valsartan and candesartan, a concluding hydrolysis step is typically required for the production of the desired end product, before the desired product can be obtained as a pure substance or salt. A particularly detailed description of the preferred reactions can be found in EP 796852. In particular, when using organotin compounds, one should take into consideration that they are highly toxic substances, whose quantitative separation is an essential prerequisite for the applicability of the product obtained. Dealing with azides in organic solvents requires a number of safety precautions; in particular, the concluding step of an acidification following hydrolysis can lead to the formation of highly explosive quantities of hydrazoic acid, wherein there is also a great explosion risk in addition to the high toxicity.

The goal of the invention under consideration is to design this essential concluding step in the synthesis of 5-substituted tetrazoles, mentioned above, in a manner that provides greater safety and guarantees quantitative separation of the starting product and reactants in the concluding purification step and in particular, before the acidification. To attain this goal, the method in accordance with the invention essentially consists of first mixing the organic phases containing the nitrile and the tetrazole with water, forming three liquid phases, after which the aqueous phase containing the azide and the upper phase containing the nitrile are separated out, and the middle organic phase containing the tetrazole is subsequently treated. In the case of ester groups to be saponified, this phase is mixed with alkali lye, after which the organic phase is separated out and the aqueous phase is acidified or, otherwise, this phase is immediately acidified and purified. If the 5-substituted tetrazoles fulfill certain conditions with respect to hydrophilic and lipophilic substituents, and in particular, if the substituted biphenyl radicals are 5-substituted tetrazoles, immediate hydrolysis is not necessary, for example, after the reaction of the azide with the nitrile in the presence of amine salts, such as triethylamine hydrochloride, but rather water is first added so as to form three liquid phases. Whereas the reaction also takes place initially in three phases making up a solid-liquid-liquid system, it is also possible to dissolve the solid phase after the end of the reaction by the addition of water. It has been surprisingly shown that one of the two liquid phases already present clearly expands. In principle, the organic liquid phases consist of the solvent, in particular an aromatic solvent, especially toluene, xylene, or mesitylene; this solvent, of course, contains the nonreacting starting product, namely, the corresponding nitrile, and impurities, if they are soluble in this solvent. The water-soluble components of the reaction mixture and, in particular, the originally solid phase, are found in the aqueous phase, which now contains nonreacted sodium azide and triethylamine hydrochloride, for example. An expanding middle phase with the organic solvent containing the desired product, namely, the 5-substituted tetrazole in a high concentration, is then formed between these two phases. This step, which is upstream from the subsequent purification or, if necessary, the hydrolysis step, in which the mixture is mixed with water, thus permits performance of a high degree of preliminary purification in a particularly simple manner; in particular, nonreacted azides can be discharged with the aqueous phase. In one single step, therefore, a highly concentrated 5-substituted tetrazole can be freed from nonreacted educt/intermediate product and some impurities present in small quantities, wherein the separation of the salts is essential, not least because in the case of a nonseparation during the acidification, large quantities of hydrazoic acid are released and thus, in addition to the high toxicity, there would also be a high explosion risk.

As is proposed in accordance with the invention, the middle organic phase can be subsequently mixed with alkali lye, so that, depending on the type of substituents, a saponification or hydrolysis can be carried out in case the compound present in the middle organic phase is not the end product.

As already mentioned above, the 5-substituted tetrazoles are preferably compounds of general formula I, in which R represents a substituted biphenyl radical. In accordance with the invention, the concretely defined compounds valsartan, losartan, irbesartan, candesartan, and olmesartan are particularly preferred. In the case of valsartan, the nitrile is N-valeryl-N-[(2′-cyanobiphenyl-4-yl)methyl]-(L)-valine methyl ester, which of course must be subsequently saponified to obtain the end product, namely, (S)-N-(1-carboxy-2-methylprop-1-yl)-N-pentanoyl-N-[2′-(1H-tetrazol-5-yl)biphenyl-4-ylmethyl]amine.

In the case of valsartan, it should be noted that in the reaction of N-valeryl-N-[2′-cyanobiphenyl-4-yl)methyl]-(L)-valine methyl ester with alkali azides, the alkali metal salt of (S)-N-(1-methoxycarboxy-2-methylprop-1-yl)-N-pentanoyl-N-[2′-(1H-tetrazol-5-yl)biphenyl-4-ylmethyl]amine is formed, which, because of its special characteristics (lipophilic substituents with the simultaneous presence of an ionic group), dissolves neither in water nor in toluene, but rather is deposited in the interphase in a highly concentrated form and as a third phase.

In a subsequent reaction to attain the purified end product, the middle organic phase containing the highly concentrated and still esterified product is subjected to a hydrolysis or saponification with aqueous or ethanolic potassium hydroxide or sodium hydroxide, after which an organic and an aqueous phase form. The lower phase, which is aqueous for the most part, is subsequently treated and then contains the saponified or hydrolyzed product, whereas the upper phase containing the selected solvent, for example, toluene, xylene, or mesitylene, is discarded.

In the further workup, the separated aqueous phase is preferably mixed and acidified, subsequently, with an organic solvent, preferably, a lower acetic acid alkyl ester such as methyl acetate, ethyl acetate, or butyl acetate. Here, it is essential that this aqueous phase does not include any azides, after which, while heating, branched or cyclic hydrocarbons and/or ethers, in particular methylcyclohexane and/or diisopropyl ether are added. A 1-2 quantitative ratio of acetic acid ester to the subsequently added branched or cyclic hydrocarbon or diisopropyl ether has proved good here. Later, the organic phase is treated further, and water is separated out completely by means of a water separator. The complete separation of water is a prerequisite for obtaining a partially crystalline, filterable product in the following crystallization process. Even small quantities of water will lead to a two-phase system, in which the product separates as a second liquid phase and cannot be filtered. After the cooling and crystallizing out of the product, the product can be separated out by filtration in a simple manner, and dried.

The invention is explained in more detail below with the aid of examples.

### EXAMPLE 1

### Production of Valsartan

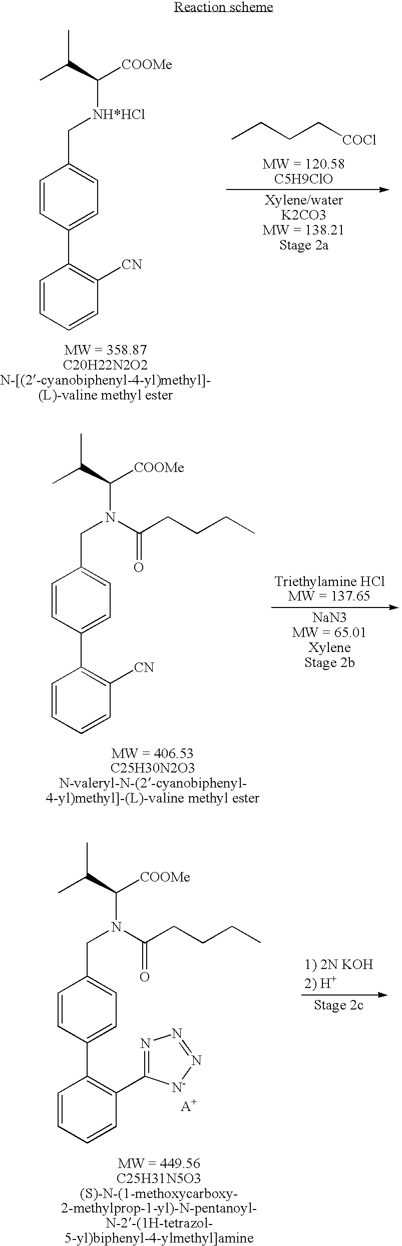

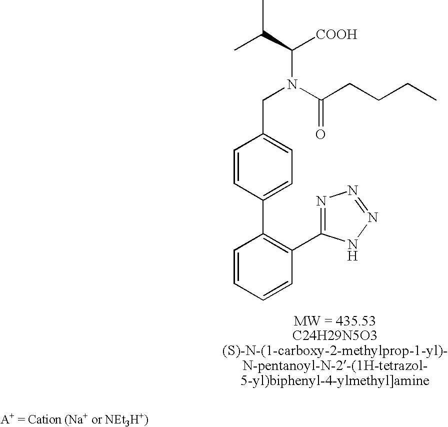

First, K₂CO₃ (110 g) is dissolved in water (250 mL). Then, toluene (800 mL) and N-[(2′-cyanobiphenyl-4-yl)methyl]-(L)-valine methyl ester (100 g) are added, followed by vigorous stirring at room temperature until all solids have dissolved (approx. 30 min).

Valeroyl chloride (44 mL) is added dropwise, at T<20° C. Subsequently, stirring is carried out for 1.5-2.0 h, at 20-25° C. Salts which precipitate during the reaction are filtered off.

The aqueous phase is separated; the organic phase is washed with a mixture of 100 mL brine and 100 mL water; the washing phase is separated and discarded.

Sodium azide (54 g) and triethylamine hydrochloride (115 g, each 3.0 Eq) are added; subsequently, stirring is carried out for 20-24 h, at 90±3° C. Before the subsequent addition of water, a three-phase system (solid-liquid-liquid) is present. The two liquid phases correspond to the upper and middle phases with the subsequent addition of water, which apparently increases the volume of the middle phase.

Water (250 mL) is added, followed by vigorous stirring, until all solids have dissolved. 3 phases. The lower phase is discarded; the two upper phases are washed with 200 mL water; the washing phase and the upper phase are discarded; and the middle phase is used for the further treatment.

The uppermost phase (toluene) contains nonreacted N-[(2′-cyanobiphenyl-4-yl)methyl]-(L)-valine methyl ester and N-valeryl-N-[(2′-cyanobiphenyl-4-yl)methyl]-(L)-valine methyl ester and impurities; has a light appearance; and is light brownish-yellow;

The middle phase (toluene and a small amount of water) contains highly concentrated (S)-N-(1-methoxycarboxy-2-methylprop-1-yl)-N-pentanoyl-N-[2′-(1H-tetrazol-5-yl)biphenyl-4-ylmethyl]amine solution and is brown in appearance.

The lower phase (aqueous) contains salts (nonreacted sodium azide and triethylamine hydrochloride) and is light brownish-yellow in appearance.

By means of this three-phase system, it is possible in one step to free (S)-N-(1-methoxycarboxy-2-methylprop-1-yl)-N-pentanoyl-N-[2′-(1H-tetrazol-5-yl)biphenyl-4-ylmethyl]amine both of salts as well as of nonreacted educt/intermediate product and some impurities present in small quantities. The separation of the salts is essential, because in case of a nonseparation during the concluding acidification, large quantities of hydrazoic acid (HN₃) would be released (high toxicity and explosion risk).

The addition of 14% (2.5N) potassium hydroxide (400 mL) to the isolated middle phase is carried out, whereupon stirring is performed for 3.0 h at 40±3° C.

2 phases form. A lower phase, which is aqueous for the most part, is ((S)-N-(1-carboxy-2-methylprop-1-yl)-N-pentanoyl-N-[2′-(1H-tetrazol-5-yl)biphenyl-4-ylmethyl]amine) with a small volume of a toluene upper phase. The upper phase is separated and discarded.

5 g activated carbon and 5 g celite are added to the lower phase and stirring is carried out for 1 h at 40-50° C., after which filtration is performed. Then, 720 mL ethyl acetate are added and acidification to pH 2.0±0.5 is carried out with 6N HCL. The aqueous lower phase is separated, the organic upper phase is washed with 200 mL water, and the aqueous phases are discarded.

Subsequently, heating to 50° C. is carried out and 480 mL methylcyclohexane are added dropwise.

Water is completely separated out with a water separator. A complete water separation is indispensable (the prerequisite for the crystallization in the following step). The presence of even small quantities of water leads to a two-phase system, where the product can separate as a second liquid phase and cannot be filtered. Cooling is carried out slowly to 5±5° C., followed by stirring for 1 h, filtering, and washing with ethyl acetate-methylcyclohexane 3/2, whereupon drying is performed at 40° C. in a vacuum.

Yield: approx. 65% over all stages.

In general, for the sartans cited in the following, it is accepted that three-phase liquid systems typically can be expected in the workup. In the case of candesartan, a methyl ester group is present, just as with valsartan, which is split to the free acid by hydrolysis.

What is valid in principle is that if a carboxylic acid ester is converted into a free acid, one can speak correctly of a synthesis, whereas in other cases in which such an ester splitting is not required in the last step, one can make reference only to a purification, strictly speaking. The concluding step of the hydrolysis with subsequent acidification is, however, in any case to be understood as a purification step also, so that the selected nomenclature of pure preparation makes no difference here between purifying and synthesizing. In another embodiment example of the pure preparation of valsartan via hydrolysis by means of aqueous KOH, it was possible to increase the yield, over the last stage, to approximately 75% of the theoretical yield.

### EXAMPLE 2

### Synthesis of Valsartan (Hydrolysis by Means of Aqueous KOH)

Stages 2b and 2c in the reaction scheme above.

N-valeryl-N-[(2′-cyanobiphenyl-4-yl)methyl]-(L)-valine methyl ester (110 g, 270 mmol) is reacted in an aromatic hydrocarbon, preferably toluene, xylolene, or mesitylene (typically, 500-1000 mL), with alkali metal azides and another reagent (ammonium halide derivatives, typically, triethylamine hydrochloride, or organotin halides, typically, trimethyltin chloride or tributyltin chloride), while heating, to form (S)-N-(1-methoxycarboxy-2-methylprop-1-yl)-N-pentanoyl-N-[2′-(1H-tetrazol-5-yl)biphenyl-4-ylmethyl]amine. The initial solid-liquid two-phase system is converted, as the reaction progresses, into a three-phase system (solid-liquid-liquid).

After completion of the reaction, the reaction solution is stirred with water or a saline solution (250 mL), whereupon the solids dissolve and a three-phase liquid system forms. The lower phase is separated; the two upper phases are washed with water or a saline solution (200 mL). The middle phase is isolated and stirred vigorously with aqueous potassium hydroxide (2.5N, 400 mL) for 3 h at 40° C. A two-phase system forms with an aqueous, product-containing lower phase and an organic upper phase. The aqueous phase is isolated, stirred with 5 g activated carbon and 5 g celite for 1 h at 40° C., and then filtered. Ethyl acetate (720 mL) is added to the filtrate and acidification is carried out with hydrochloric acid (5-6N) to pH 2.0, with vigorous stirring and ice cooling. The organic phase is washed with 300 mL water and after separation of the washing phase, an aliphatic hydrocarbon or a mixture of aliphatic hydrocarbons (480 mL) is added dropwise, preferably methylcyclohexane or isooctane. The residual water present in the system is separated out by means of a water separator. Cooling is done slowly to 5° C., at which point crystallization begins. The solids are filtered off, washed with a mixture of ethyl acetate and hydrocarbon and dried at 40° C. in a vacuum.

Yield over both stages: approx. 75% of the theoretical.

### EXAMPLE 3

### Synthesis of Candesartan (Hydrolysis by Means of Ethanolic KOH)

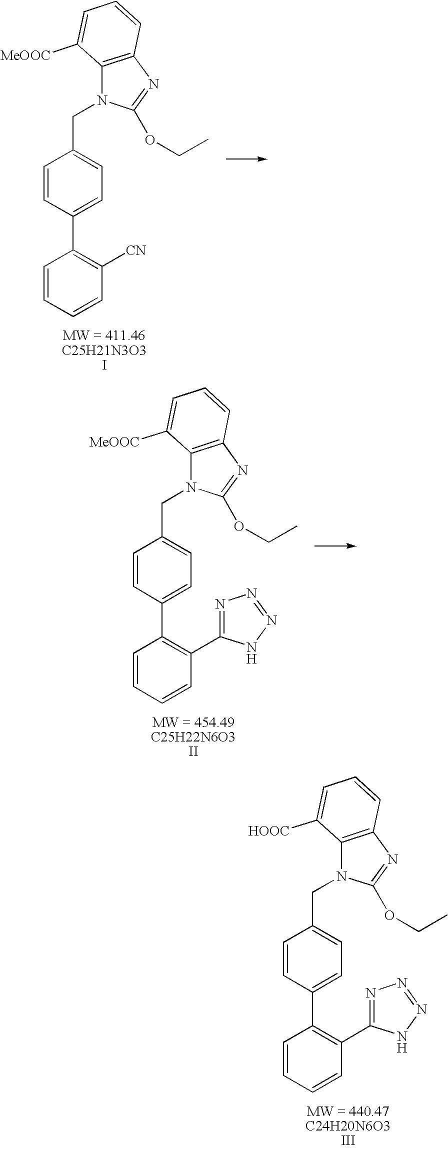

Compound I, 1-(2′-cyanobiphenyl-4-yl)methyl)-2-ethoxybenzimidazole-7-carboxylic acid methyl ester (111 g, 270 mmol), is reacted in an aromatic hydrocarbon, preferably toluene, xylolene or mesitylene (typically, 500-1000 mL), with alkali metal azides and another reagent (ammonium halide derivatives, typically, triethylamine hydrochloride or organotin halides, typically, tetramethyltin chloride or tetrabutyltin chloride), while heating, to form compound II, 2-ethoxy-1-((2′-(1H-tetrazol-5-yl)biphenyl-4-yl)methyl)benzimidazole-7-carboxylic acid methyl ester. After completion of the reaction, the reaction solution is stirred with water or a saline solution (250 mL), whereupon the solids dissolve and a three-phase liquid system forms. If only two phases are present, petroleum spirit 80/110 is added until there are three phases which can be separated well. The lower phase is separated; the two upper phases are washed with water or a saline solution (200 mL). The middle phase is isolated and stirred with potassium hydroxide in ethanol (2.5N, 400 mL) for 2 h at 40° C. Water (400 mL) is added, and 500 mL liquid are distilled off under reduced pressure. With the addition of 5 g actived carbon and 5 g celite, stirring is carried out for 1 h at 40° C., followed by filtration. Ethyl acetate (720 mL) is added to the filtrate and acidification is carried out with hydrochloric acid (5-6N) to pH 2.0, while stirring vigorously and with ice cooling. The organic phase is washed with 300 mL water, and after separation of the washing phase, an aliphatic hydrocarbon or a mixture of aliphatic hydrocarbons (480 mL) is added dropwise, preferably, methylcyclohexane or petroleum spirit 80/110. The residual water present in the system is separated out by means of a water separator. Cooling is done slowly to 5° C., at which point crystallization begins. The solids are filtered off, washed with a mixture of ethyl acetate and hydrocarbon, and dried at 40° C. in a vacuum.

Yield over both stages: approx. 70% of the theoretical.

### EXAMPLE 4

### Synthesis of Valsartan (Hydrolysis by Means of Tetraalkylammonium Hydroxide Bases)

Stages 2b and 2c in the reaction scheme above.

N-valeryl-N-[(2′-cyanobiphenyl-4-yl)methyl]-(L)-valine methyl ester (110 g, 270 mol) is reacted in an aromatic hydrocarbon, preferably toluene, xylolene or mesitylene (typically, 500-1000 mL), with alkali metal azides, and another reagent (ammonium halide derivatives, typically, triethylamine hydrochloride or organotin halides, typically, trimethyltin chloride or tributyltin chloride), while heating, to form (S)-N-(1-methoxycarboxy-2-methylprop-1-yl)-N-pentanoyl-N-2′-(1H-tetrazol-5-yl)biphenyl-4-ylmethyl)amine. The initial solid-liquid two-phase system is converted, as the reaction progresses, into a three-phase system (solid-liquid-liquid).

After completion of the reaction, the reaction solution is stirred with water or a saline solution (250 mL), whereupon the solids are dissolved and a three-phase liquid system forms. The lower phase is separated; the two upper phases are washed with water or a saline solution (200 mL). The middle phase is isolated and stirred with tetrabutylammonium hydroxide 40% in methanol (260 mL, 400 mmol) for 3 h at 40° C. Water (400 mL) is added, and 400 mL liquid are first distilled off under normal pressure and, toward the end, under reduced pressure. With the addition of 5 g activated carbon and 5 g celite, stirring is carried out for 1 h at 40° C. followed by filtration. Ethyl acetate (720 mL) is added to the filtrate, followed by acidification with hydrochloric acid (5-6N) to pH 2.0, while stirring vigorously and with ice cooling. The organic phase is washed twice with 300 mL water and after separation of the washing phase at approx. 50° C., an aliphatic hydrocarbon or a mixture of predominantly aliphatic hydrocarbons (480 mL) is added dropwise, preferably, methylcyclohexane or petroleum spirit 80/110. The residual water present in the system is separated out by means of a water separator. Cooling is carried out slowly to 5° C., at which point crystallization begins. The solids are filtered off, washed with a mixture of ethyl acetate and hydrocarbon and dried at 40° C. in a vacuum. Yield over two stages, each according to the synthesis protocol of II: approx. 70% of the theoretical.

### EXAMPLE 5

### Synthesis of (S)-N-(1-carboxy-2-methylprop-1-yl)-N-[2′(1H-tetrazol-5-yl)biphenyl-4-yl methyl]amine Hydrochloride (Compound V)

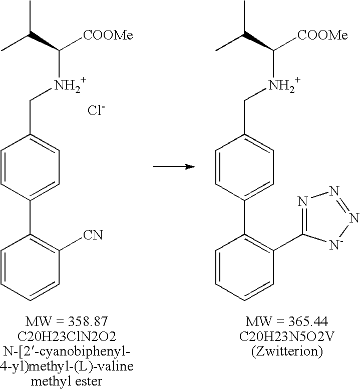

N-[(2′-cyanobiphenyl-4-yl)methyl]-(L)-valine methyl ester (96.9 g, 270 mmol) is reacted in an aromatic hydrocarbon, preferably toluene, xylolene or mesitylene (typically, 500-1000 mL), with alkali metal azides and another reagent (ammonium halide derivatives, typically, triethylamine hydrochloride, or organotin halides, typically, trimethyltin chloride or tributyltin chloride), while heating, to form (S)-N-(1-methoxycarboxy-2-methylprop-1-yl)-N-pentanoyl-N-[2′-(1H-tetrazol-5-yl)biphenyl-4-ylmethyl]amine. The initial solid-liquid two-phase system is converted, as the reaction progresses, into a three-phase system (solid-liquid-liquid).

After the completion of the reaction, water (200 mL) is added. The solids are thereby dissolved. Subsequently, the pH is adjusted to 6-7, whereupon a three-phase liquid system forms. The lower phase is separated; the two upper phases are washed with water (200 mL). The middle phase is isolated, mixed with ethyl acetate (500 mL), washed with water (200 mL), dried with sodium sulfate and filtered. The solvent is evaporated on a rotavapor; the product is dried at 60° C. in a vacuum. Yield, 58-60%.

1. A method for isolating a 5-substituted tetrazole of general formula I: 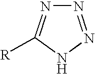
in which R represents a substituted biphenyl radical, comprising:
providing a corresponding nitrile and performing the ring closure reaction in an organic solvent while using alkali or alkaline-earth metal azides or organotin azides, and
after the ring closure reaction has been performed, the organic phases containing the nitrile and the tetrazole are first mixed with water while forming three liquid phases, after which the aqueous phase containing the azide and the phase containing the nitrile are separated out, and the middle organic phase containing the tetrazole is processed.

2. A method of claim 1, wherein the compound of general formula I is valsartan which has the following structure

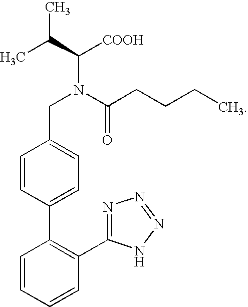

3. A method of claim 1, wherein the compound of general formula I is losartan which has the following structure

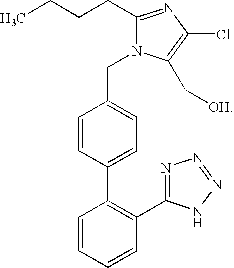

4. A method of claim 1, wherein the compound of general formula I is irbesartan, which has the following structure

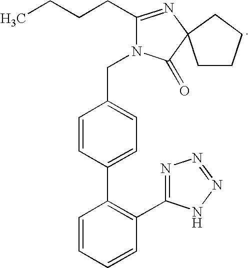

5. A method of claim 1, wherein the compound of general formula I is candesartan, which has the following structure

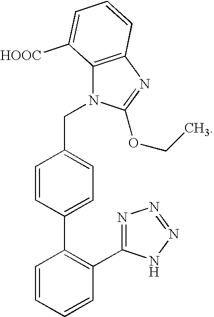

6. A method of claim 1, wherein the compound of general formula I is olmesartan which has the following structure

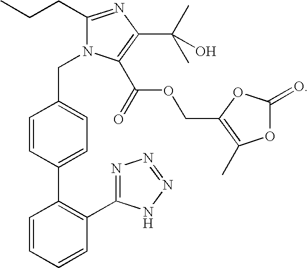

7. A method of claim 1, wherein the reaction of the nitrile of general formula R—C≡N with a metal azide of general formula M(N₃)ₙ, wherein M is an alkali or alkaline-earth metal, and n is 1 or 2, takes place in the presence of an amine salt in an aromatic solvent.

8. (canceled)

9. A method of claim 1, wherein the middle organic phase containing the tetrazole is processed by being acidified.

10. A method of claim 1, wherein the middle organic phase containing the tetrazole is processed by being mixed with alkali lye, after which the organic phase is separated out and the aqueous phase is acidified.

11. A method of claim 7, wherein the aromatic solvent is selected from the group consisting of toluene, xylene, and mesitylene.

12. A method of claim 11, wherein, the middle organic phase containing the tetrazole is processed by being mixed with aqueous or ethanolic KOH or NaOH, whereupon an organic and an aqueous phase form.

13. A method of claim 1, wherein the middle organic phase containing the tetrazole is processed by being mixed with alkali lye, after which the organic phase is separated out and the aqueous phase is acidified and extracted with ethyl acetate, and the resulting organic phase is mixed with a compound selected from the group consisting of branched hydrocarbon, cyclic hydrocarbon, and ether, and water is separated out.

14. The method of claim 13, further comprising filtering and drying the tetrazole.

15. The method of claim 13, wherein the compound selected from the group consisting of branched hydrocarbon, cyclic hydrocarbon, and ether is methylcyclohexane, diisopropyl ether, or a mixture thereof.
